(19) United States
(12) Patent Application Publication
(10) Pub. No.: US 2008/0234790 A1
(43) Pub. Date: Sep. 25, 2008

(54) Implantable Stimulation Electrode with a Coating for Increasing Tissue Compatibility

(76) Inventors: Gerd Bayer, Erlangen (DE); Alexander Borck, Aurachtal (DE)

Correspondence Address: HAHN LOESER & PARKS, LLP, One GOJO Plaza, Suite 300, AKRON, OH, 44311-1076, US

(21) Appl. No.: 10/562,095

(22) PCT Filed: May 21, 2004
(86) PCT No.: PCT/EP04/05550

§ 371 Date: Oct. 6, 2006

(30) Foreign Application Priority Data
Jun. 21, 2003 (DE) 103 28 816.3

(51) Int. Cl. A61N 1/05

(52) U.S. Cl. 607/116

(57) ABSTRACT

An implantable stimulation electrode for use with an implantable tissue stimulator, especially a pacemaker, a defibrillator, a bone stimulator or a neurostimulator includes a metal base body, optionally one or more intermediate layers disposed on the base body and a coating covering the base body and, optionally, intermediate layers in order to increase tissue compatibility. The coating should prevent tissue irritations after implantation and more particularly increase the stimulus threshold associated therewith, have very high biocompatibility and also has an anti-inflammatory effect. An increase in tissue compatibility is achieved by virtue of the fact that the coating has a polysaccharide layer made of hyaluronic acid and/or hyaluronic acid derivatives.

### BACKGROUND OF THE INVENTION

The present invention relates to an implantable stimulation electrode having a coating to increase the tissue compatibility.

Implantable electrodes for the stimulation of bodily tissue, particularly for use in pacemakers, defibrillators, and bone stimulators or neurostimulators, are known in manifold forms. The great majority of stimulation electrodes of this type are based on metallic materials, since these are predestined for the transmission of electrical currents to living tissue because of their good conductivity. Other achievements of the object provide the use of conductive polymers (e.g., U.S. Pat. No. 5,080,099).

High electrode capacitance and therefore low electrode impedance and the highest possible degree of biocompatibility are of outstanding importance for the usage value of an implantable stimulation electrode—particularly one which is intended for long-term use on a tissue stimulator having an exhaustible energy source and which therefore must contribute to the minimal energy consumption.

Thus, for example, a highly developed implantable stimulation electrode was described in EP 0 453 117 A1 and WO 93/02739. The electrode comprises a multilayered platinum base body, which is compressed from fiber or wire material, an adhesive layer, a Pt, C, or Al texturing layer having a rough surface, and a catalytic Pt or Pt/C cover layer. Furthermore, the stimulation electrode has a very large active surface having a fractal surface structure and may alternately also be implemented in the form of a titanium base body having an iridium, iridium nitrite, or iridium oxide coating.

In general, a temporary irritation threshold increase may be detected in the first weeks after the implementation of stimulation electrodes, which is to be attributed to local occurrences of inflammation of the adjoining tissue. These occurrences of inflammation additionally result in unfavorable ingrowth behavior of the stimulation electrodes, which has a negative influence on the long-term stimulation properties of the system.

An implantable stimulation electrode of the type according to the species is known from U.S. Pat. No. 5,964,794, the disclosure of which is incorporated by reference herein in connection with the present invention. The stimulation electrode described therein particularly displays increased tissue compatibility. This is achieved in that a thin, specifically functionalized organic coating, which forms essentially the entire outer surface of the stimulation electrodes, is provided, which adheres permanently to the surface underneath because of reversible physisorption or covalent bonding. Among other things, silane and synthetic polymers such as polystyrene sulfonate, polyvinyl sulfonate, or polyallyl amine are suggested as coating materials. The organic coating may also be multilayered, polyethylene oxide or polyethylene glycol being terminated on the external surface in particular. Furthermore, it is claimed that the organic layer contains a medicinal active ingredient, in particular an anti-inflammatory medication, which may be administered from the organic coating controlled by diffusion or solution.

The improvements described through the coating of the stimulation electrode do result in a significant reduction of the temporary irritation threshold increase, but are relatively complex and therefore costly to implement and, because of the synthetic nature of the materials used, require extensive tests for evaluating the biocompatibility. Furthermore, in the case of the desired addition of anti-inflammatory active ingredients, it is necessary to tailor the material properties of the active ingredients and the organic coating in which they are embedded to one another through extensive tests.

### BRIEF SUMMARY OF THE INVENTION

An aspect of the present invention is to provide a coating for an implantable stimulation electrode which avoids tissue irritation after the implantation and an irritation threshold increase connected therewith in particular. The coating is to have very high biocompatibility and is additionally to have an anti-inflammatory effect. Furthermore, the coating is to comprise as few components as possible, so that the production is simplified.

This aspect is achieved by the implantable stimulation electrode according to the present invention.

The implantable stimulation electrode has a coating forming essentially the entire external surface of the stimulation electrode, which adheres to the surface underneath through physisorption or covalent bonding. The coating covers the metallic base body and possibly one or more intermediate layers applied to the base body. The coating comprises a polysaccharide layer made of hyaluronic acid and/or hyaluronic acid derivatives. Surprisingly, it has been shown that the application of such a polysaccharide layer does not result in any noticeable increase of the electrode impedance and accordingly has hardly any or no influence on the energy consumption of the stimulation electrode. Furthermore, hyaluronic acid and its derivatives are distinguished by their very good biocompatibility, since the materials are of natural origin. In addition, it has been shown that hyaluronic acid and its derivatives have an intrinsic anti-inflammatory effect and therefore may effectively prevent or at least strongly reduce tissue irritation.

Hyaluronic acid (hyaluronan) is a simple glycosaminoglycan of the extracellular matrix. It is synthesized on the surface of fibroblasts and occurs as a single glycosaminoglycan, not as a proteoglycan. Hyaluronic acid is a high-molecular-weight compound having M_{R} between 50,000 and several million. The basic component of hyaluronic acid is an aminodisaccharide, synthesized from D-glucuronic acid and N-acetyl-d-glucosamine in β1-3-glycosidic bonding, which has a β1-4-glycosidic bond to the next unit:

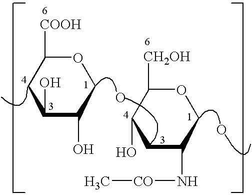

The unbranched chain of hyaluronic acid comprises 2,000-10,000 such units. β-glycosidic bonds are hydrolyzed through hyaluronidase and the hyaluronic acid is thus decomposed into smaller fragments. Commercially available hyaluronic acid—usually as a potassium salt—is isolated from human umbilical cords or cockscombs, but is increasingly manufactured in biotechnology through bacterial fermentation.

Methods known from the literature are used for modifying hyaluronic acid, i.e., preparing hyaluronic acid derivatives (e.g., Danishefsky, Arch. Biochem. Biophys., 90, 1960, p. 114 et seq.; Nagasawa, Carbohydr. Res., 58, 1977, p. 47 et seq.; Ayotte, Carbohydr. Res. 145, 1986, p. 267 et seq.; Ogamo, Carbohydr. Res. 193, 1989, p. 165 et seq.; Jesaja, Can. J. Chem.; 67, 1989, p. 1449 et seq.; Mulloy, Carbohydr. Res. 255, 1994, p. 1 et seq.). These are regioselective and stereoselective and non-regioselective and non-stereoselective (static) reactions. Based on these methods, hyaluronic acid may particularly be altered through N and O desulfation, O desulfation, 6-O desulfation, deacetylation, or acetylation, as well as sulfation and acylation with aliphatic or aromatic residues. In particular, through the known methods, amino groups and sulfate or carboxyl residues may be introduced by using protective group chemistry and known, partially regioselective reactions of organic chemistry.

As defined in the present invention, the term “hyaluronic acid derivatives” is understood to include all reaction products which are structurally changed from the starting product through targeted modifications of natural hyaluronic acid. Furthermore, the term “hyaluronic acid and hyaluronic acid derivatives” is understood to include all polyelectrolytic salts thereof, e.g., sodium, potassium, magnesium, and potassium salts. The listed reactions and further known reactions of organic chemistry for reacting the functional groups of hyaluronic acid are considered “modifications” as defined in the present invention.

Hyaluronic acid and the hyaluronic acid derivatives may be immobilized on the stimulation electrode surface covalently and/or through physisorption as individual substances, copolymers or block polymers of hyaluronic acid and hyaluronic acid derivatives, and also in the form of mixtures of the above-mentioned individual substances and polymers.

Covalent bonding of the polysaccharide layer to the surface of the stimulation electrode is preferably performed through single-point or multipoint suspension on spacers. Furthermore, mechanical and/or chemical stabilization of the coating material against enzymatic and hydrolytic degradation and also against mechanical stress is preferably achieved through cross-linking of a previously applied (primary) polysaccharide layer. The immobilization of the polysaccharide layer on the surface of the stimulation electrode may be performed according to known methods of immobilization of enzymes, methods of membrane manufacturing, plastic processing, polymer chemistry, peptide, protein, and sugar chemistry via covalent bonds with and without the use of spacers, using single point and multipoint suspension, and point suspension as a monolayer or multilayer or with additional stabilization through cross-linking.

A coating having a layer thickness in the range between 10-400 μm, particularly 50-120 μm, has been shown to be advantageous. At the cited layer thicknesses, no significant effect on the functionality of the stimulation electrode could be determined.

Furthermore, the hyaluronic acid or the hyaluronic acid derivatives may have an average molecular weight in the range from approximately 300,000-500,000, particularly 380,000-420,000 g/mole after sterilization. The intrinsic therapeutic effect of hyaluronic acid and its derivatives reach a maximum in the claimed molecular weight range (Papkonstantinou, G. Karakulakis, O. Eickelberg, A. P. Perruchoud, L. H. Block, and M. Roth; A 340 kDa hyaluronic acid secreted by human vascular smooth muscle cells regulates their proliferation and migration, Glycobiology 1998, 8, 821-830).

A further advantageous aspect of the teaching according to the present invention is the targeted influencing of the in vivo degradation behavior of the biopolymer. The term “degradation behavior” is understood to include degradation of the polysaccharide layer according to the present invention occurring through chemical, thermal, oxidative, mechanical, or biological processes in the living organism over time. It is to be ensured that at least in the first weeks after the implantation, local occurrences of inflammation of the adjoining tissue are reduced or even avoided. However, the coating is to prevent or at least significantly suppress surface adsorption of high-molecular-weight biomolecules on the stimulation electrode over a specific period of time, since otherwise an increase of the electrode impedance is also to be expected in the moderate and long term.

The polysaccharide layer may have a composition such that the in vivo degradation of the polysaccharide layer is slowed from the outside in the direction of the base body of the implant. The degradation behavior may be altered continuously or suddenly in this case. According to the latter variation, the polysaccharide layer comprises at least two partial layers having different degradation behaviors, the degradation behavior within each partial layer being able to be fixed as continuously changeable or constant over the partial layer. The manufacturing of coatings of this type may be performed with the aid of spray and immersion coating methods known per se.

The polysaccharide layer may have a composition such that an external area of the polysaccharide layer, which faces away from the base body of the electrode, is degraded within 100 days in vivo. The external area may be 10 to 250 μm, particularly 50 to 150 μm thick. If the polysaccharide layer comprises at least two partial layers having different degradation behaviors, to achieve this goal, an external partial layer may be modified in such a way that this external partial layer is degraded by more than 50 weight-percent within 100 days in vivo. The external partial layer may be 10 to 250 μm, particularly 50 to 150 μm thick.

Surprisingly, it has also been shown that in the presence of the polysaccharide layer according to the present invention, the surface adsorption of high-molecular-weight biomolecules on the electrode surface is also prevented or at least significantly repressed. Therefore, the polysaccharide layer preferably has a composition such that an internal area of the polysaccharide layer, which faces toward the base body of the electrode, is not completely degraded within two years in vivo. The internal area may be 3 to 50 μm, particularly 5 to 20 μm thick. If the polysaccharide layer comprises at least two partial layers having different degradation behaviors, to achieve this goal, an internal partial layer, which directly adjoins the surface of the base body underneath it or possibly an intermediate layer applied thereto, may be particularly modified in such a way that this internal partial layer is not degraded by more than 20 weight-percent within two years. The external partial layer may be 3 to 50 μm, particularly 5 to 20 μm thick.

The degradation behavior of hyaluronic acid and its derivatives may be influenced by cross-linking, among other things. For this purpose, reference is made in general to the numerous methods described in the literature for performing the individual cross-linking reactions and expressly to the objects of U.S. Pat. No. 4,582,865, U.S. Pat. No. 5,550,187, U.S. Pat. No. 5,510,121, and WO 00/46252. Reductive fixing is understood as the targeted reaction of unsaturated functionalities of the polysaccharide with hydridic reduction agents, such as sodium borohydride. For example, cross-linking may be performed with the aid of the following reagents:

Formaldehyde, glutaraldehyde, divinyl sulfone, polyanhydrides, polyaldehydes, carbodiimides, epichlorohydrin, ethylene glycol diglycidyl ether, butane diol diglycidyl ether, polyglycerol polyglycidyl ether, polyethylene glycol diglycidyl ether, polypropylene glycol diglycidyl ether, or bis or polyepoxy cross-linking agents, such as 1,2,3,4-diepoxybutane or 1,2,7,8-diepoxyoctane.

The relationship between degree of cross-linking and degradation behavior may be determined via typical testing methods. A differing degree of cross-linking results in a differing swelling behavior of the polysaccharide layer. The swelling behavior may be determined gravimetrically, among other things. Furthermore, the degree of cross-linking may also be determined through infrared spectroscopic analysis of cross-linked hyaluronic acid films. The reference for degradation may be produced through a GPC analysis, i.e., through molar mass determination of degraded hyaluronic acid, on eluents.

The influence of the cited modifications on the in vivo degradation behavior is generally known. However, since the degradation behavior is a function of further geometric and physiological factors, among other things, individual adaptation of the system to the particular requirements is typically necessary.

The coating may typically be applied to all known stimulation electrodes. The thin polysaccharide layer made of hyaluronic acid and/or hyaluronic acid derivatives is deposited using typical spraying methods or from solution for this purpose.

The manufacturing of a covalently adhering polysaccharide layer is described in principle in WO 00/56377, whose disclosure is incorporated herein by reference in its entirety. A substrate surface is modified with reactive functionalities for this purpose, activated hyaluronic acid is provided, and this is then bound covalently to the reactive functionalities under suitable conditions. The polysaccharide layer according to the present invention may be bound to the surface of the stimulation electrode in the same way.

Furthermore, DE 196 30 563 (U.S. Pat. No. 5,964,794) discloses a method for improving the adhesion of a coating as a result of reinforced physisorption and/or covalent binding. In a first step, a reactive functionality is produced on the substrate surface. The reactive functionality particularly comprises amines, aldehydes, sulfides, alcohols, acid halogenides, and isocyanates. The polysaccharide layer according to the present invention may then be bound covalently—using coupling methods known per se—to the cited functionality.

According to a further aspect of the present invention, the already existing intrinsic therapeutic effect of the hyaluronic acid is supplemented by further active ingredients which are embedded in the coating and are released by the gradual degradation of the coating and/or by diffusion into the surrounding tissue. It has been shown that the anti-inflammatory steroids dexamethasone and/or dexamethasone sodium phosphate (DMNP) are especially suitable for this purpose in a concentration sufficient to unfold their pharmacological effects, since they have been shown to stabilize the macrophages adjoining the implant and therefore improve the long-term compatibility of the coating.

Surprisingly, it has additionally been shown that dexamethasone and/or DMNP has a positive effect on the phase boundary capacitance of the electrode. The phase boundary capacitance, which is only insignificantly increased by the coating, is reduced nearly to the value measurable without a polysaccharide layer through the inclusion of dexamethasone and/or DMNP.

Furthermore, the polysaccharide layer may comprise an adhesion-promoting layer made of chitosan. The adhesion-promoting layer directly adjoins the base body and possibly the intermediate layer applied thereto. Surprisingly, it has been shown that very uniform and strongly adhering coatings may be produced in the presence of such an adhesion-promoting layer. In addition, chitosan is a material of natural origin and therefore has good biocompatibility. The adhesion-promoting layer may be 0.1 to 50 μm, particularly 1 to 10 μm thick and may be modified like the hyaluronic acid and its derivatives to influence its degradation behavior. In particular, the adhesion-promoting layer may be implemented in such a way that it may act as the internal partial layer or internal area of the polysaccharide layer in the above-mentioned definition. A significant change of the electrical transmission properties of the electrode by the adhesion-promoting layer was not established.

According to a further preferred variation of the present invention, the polysaccharide layer contains chitosan in at least partial areas or partial layers. In this way, the adhesive capability of the polysaccharide layer may be improved further and uniform coatings may be produced on the often very complex geometries of the substrates. The stability of the polysaccharide layer may be increased if polycationic charges are produced through quaternization of the amine functions of the chitosan. If hyaluronic acid and/or its derivatives is added as a polyanionic preparation, a Symplex gel forms. The ion/ion interaction between the components, which is already very strong, may be increased further through cross-linking. A weight component of the chitosan of the total weight of the polysaccharide layer is not more than 50% in one embodiment.

### BRIEF DESCRIPTION OF THE SEVERAL VIEWS OF THE DRAWINGS

In the following, the present invention is explained in greater detail on the basis of exemplary embodiments and the associated drawing.

FIG. 1 shows a cross-section and an enlarged detail therefrom of the distal end of a unipolar pacemaker electrode line having an electrode head according to an embodiment of the present invention and

FIG. 2 shows a schematic illustration to explain the construction of an implantable stimulation electrode according to an embodiment of the present invention.

### DETAILED DESCRIPTION OF THE INVENTION

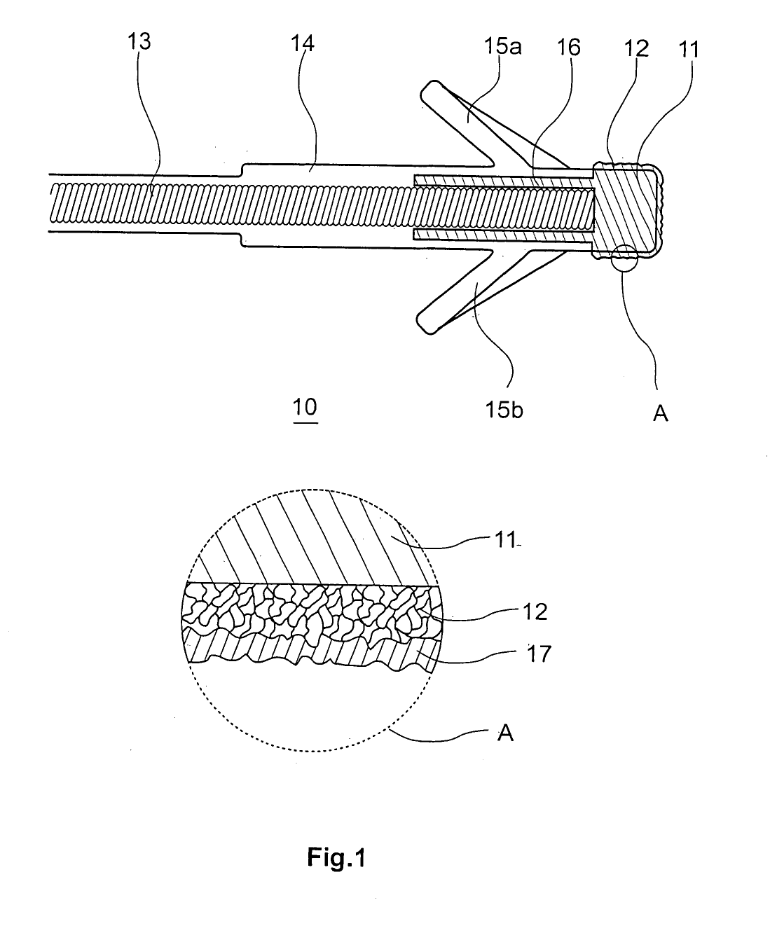

FIG. 1 schematically shows a cross-section and an enlarged detail A of the distal end of a unipolar pacemaker electrode line for a knurled electrode 10 having a cylindrical base body 11 made of titanium. The cylindrical base body 11 has a surface coating 12 (intermediate layer) comprising iridium nitrate (IrN), which is applied to the base body 11 in a known way using cathode sputtering (reactive sputtering). Furthermore, the electrode 10 comprises a coiled, electrically conductive supply line 13 having an electrically insulating sheathing 14 made of silicone. Proximally directed fins 15a and 15b are molded onto the silicone sheathing 14, which are used for fixing the electrode 10 in the heart, the surface of the base body II being held in contact with a heart inner wall (not shown here). The base body 11 is pushed over the supply line 13 using a hollow-cylindrical projection 16 and attached there.

The detail A illustrated in FIG. 1 shows a very schematic and enlarged section through an active surface of the base body 11 and its surface coating 12 and an adjoining coating 17 for increasing the tissue compatibility, which comprises at least one polysaccharide layer made of hyaluronic acid and/or hyaluronic acid derivatives. A surface of the iridium nitrate coating 12 is enlarged in a fractal way by the manufacturing, i.e., as a result of suitable selection of the method parameters of the sputtering method, the surface enlargement is from two to more than three orders of magnitude in relation to the surface of a smooth cylinder having the dimensions of the base body 11. The polysaccharide layer, which is a few to several hundred micrometers thick, is bonded to the iridium nitrate coating 12—as will be explained in greater detail in the following—the electrical properties of the electrode 10 being practically unimpaired, but the irritation threshold curve during the ingrowth of the electrode 10 in the heart wall being significantly positively influenced.

### Exemplary Embodiment 1

### Covalent Bonding

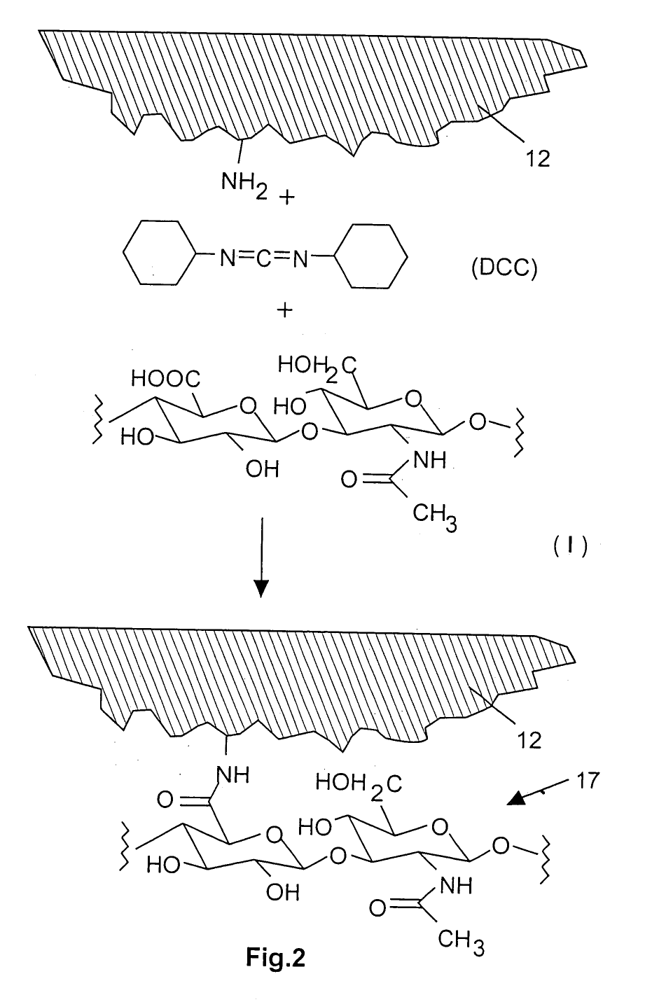

FIG. 2 discloses a schematic illustration of the construction and the preparation of a coating 17 made of hyaluronic acid, this coating being covalently bonded to the surface underneath, i.e., specifically the iridium nitrate coating 12. Alternatively or additionally, the bonding may be performed through physisorption of the hyaluronic acid on the iridium nitrate coating 12. Physisorption is understood as any electrostatic interaction between the surface of the iridium nitrate coating 12 and the hyaluronic acid (I), in particular Van der Waals interaction.

In a first method step (not shown here), amination of the iridium nitrate surface 12 is performed. Numerous known methods may be used for this purpose, primary or secondary amines, but preferably primary amines, being fixed on the surface of the iridium nitrate coating 12. Plasma activation in the presence of amines, e.g., N-heptyl amine or other aliphatic or aromatic amines, particularly suggests itself. Because of the general awareness, accessibility, and variability of methods of this type, specifics in the bonding of functionalities to the iridium nitrate coating 12 are dispensed with. It is only to be noted that reactive functionalities—a primary amine here, for example—are bound to the surface of the iridium nitrate coating 12 after the first method step has ended.

In a following, second method step, covalent bonding of hyaluronic acid (I) is performed analogously to the carbodiimide method known from peptide synthesis. Cyclohexyl carbodiimide (DCC) is specified as the coupling reagent here as an example. After establishing a peptide bond, the hyaluronic acid is covalently bonded to the electrode surface, especially the iridium nitrate coating 12 here. Suitable method parameters and variations for producing coating to this type may also be inferred from, among other things,

U.S. Pat. Nos. 5,527,893 and 5,585,361—the content of whose disclosure is hereby incorporated by reference herein.

### Exemplary Embodiment 2

### Immersion Coating

In addition to covalent bonding, hyaluronic acid and/or hyaluronic acid derivatives may also be applied to the electrode surface through simple immersion coating.

The electrode surface was precleaned and degreased and laid for 10 minutes at room temperature in an aqueous solution of hyaluronic acid having a molecular weight of at least 1,000,000 g/mole with light stirring. After removal and drying, the electrode was immersed for at least 2 hours at approximately 30° C. to 40° C. in a cross-linking agent solution of 2 to 4 ml glutaraldehyde in a water-acetone mixture. The cross-linking agent solution was then replaced for at least a further 2 hours. The electrode was then washed multiple times using distilled water and reductively fixed using a diluted solution of sodium cyanoborohydride and washed multiple times using deionized water. After removal, the sample was dried for 24 hours at 50° C. in the drying cabinet.

The molecular weight of the hyaluronic acid is to be above 1,000,000 g/mole, since the hyaluronic acid chains are cleaved by the sterilization. According to the present experiments, 1 to 2 cleavages occur per chain during a sterilization with the aid of ethylene oxide or beta irradiation (electron accelerator: 4.5 mEV, 25 kGy), i.e., native hyaluronic acid is provided having a molecular weight in the magnitude of 400,000 g/mole after sterilization.

The following layer thicknesses could be achieved as a function of the concentration of the aqueous hyaluronic acid solution:

(a) with 0.25% aqueous hyaluronic acid solution: 90 μm,
(b) with 0.5% aqueous hyaluronic acid solution: 160 μm,
(c) with 1% aqueous hyaluronic acid solution: 200 μm,
(d) and with 2% aqueous hyaluronic acid solution: 145 μm.

### Exemplary Embodiment 3

### Chitosan as Adhesion Promoter

The electrode surface was precleaned, degreased, and lightly stirred for 10 minutes at room temperature in a 0.5 to 2% acetic acid solution having a chitosan concentration between 0.1% and 0.5%. The molecular weight of the chitosan was between 100,000 g/mole and 1,000,000 g/mole. The electrode was subsequently removed and dried.

Alternatively, a thin layer of chitosan could be applied to the electrode through spraying. For this purpose, a 0.5% chitosan solution was mixed into a 0.5% acetic acid solution. The precleaned electrodes were sprayed with the aid of an airgun 5 to 20 times at intervals of 15 to 30 seconds for 0.5 to 1.0 seconds, the electrodes being dried at 40° C. to 70° C. between the spraying steps. The applied layers had a layer thickness of 1 μm to 10 μm.

After drying, the electrode was laid for 10 minutes at room temperature in an aqueous solution of hyaluronic acid having a molecular weight of at least 1,000,000 g/mole with light stirring. After removal and drying, the electrode was immersed for at least 2 hours at approximately 30° C. to 40° C. in a cross-linking agent solution of 2 to 4 ml glutaraldehyde in a water-acetone mixture. The cross-linking agent solution was then replaced and the cross-linking was continued for 2 hours. The experimental conditions also resulted in cross-linking of chitosan with the glutaraldehyde. The acid-catalyzed reaction of the aldehyde with the amine of the chitosan occurred with formation of a Schiff base.

The electrode was subsequently washed multiple times using distilled water and reductively fixed with a diluted solution of sodium cyanoborohydride and washed multiple times using deionized water. The posttreatment resulted in reduction of the Schiff base and free aldehyde functions. After removal, the sample was dried for 24 hours at 50° C. in the drying cabinet.

The chitosan functioned as an adhesion promoter, since chitosan itself is poorly soluble in the neutral range (blood). In addition, chitosan exists in cross-linked form and also forms a covalent bond to the applied hyaluronic acid layer through cross-linking with the aid of the glutaraldehyde. The thin adhesion promoter layer of chitosan of 0.1 μm to 50 μm, preferably of 1 μm to 10 μm, does not result in any significant impairment of the electrical transmission properties of the electrode.

### Exemplary Embodiment 4

### Chitosan as an Additive

In addition to the polyanions hyaluronic acid and/or its hyaluronic acid derivatives, the layer may also contain polycations such as chitosan. A further functional group for the cross-linking agent glutaraldehyde is provided by the amine of the chitosan. The aldehyde function may react both with the amine function of the chitosan and also with the carbonyl and/or hydroxyl function of the hyaluronic acid. Through these reactions, the degree of cross-linking may be additionally increased and the ionic interaction between the polyanions and the polycations may be additionally reinforced. The layer system made of polyanions and polycations may be produced through alternating spraying of the electrodes using solutions in desired concentrations of chitosan, hyaluronic acid, and hyaluronic acid derivatives.

For this purpose, precleaned electrodes are alternately sprayed with an aqueous solution made of hyaluronic acid or hyaluronic acid derivative and chitosan dissolved in acetic acid. In this case, the concentration of the hyaluronic acid or hyaluronic acid derivative is 0.1% to 1%, preferably 0.2% to 0.5%. The concentration of the acetic acid is 0.1% to 2%, preferably 0.5% to 1%. The concentration of the chitosan is 0.1% to 1%, preferably 0.2% to 0.5%. The molecular weight of the hyaluronic acid or the hyaluronic acid derivatives is at least 1,000,000 g/mole and the molecular weight of the chitosan is at least 100,000 g/mole. Both solutions are applied alternately to the electrodes with the aid of a spray method at intervals of 2 seconds to 60 seconds, preferably 15 seconds to 30 seconds. The particular proportion of polyanions and polycations may be set through the selection of the concentration of hyaluronic acid and/or chitosan and the particular spray duration. The weight component of chitosan in the overall layer system is not more than 50%. The number of spraying steps determines the layer thickness of the overall layer system. Thus, with 60 spray steps having a spray duration of 0.5 seconds, layer thicknesses between 5 μm and 10 μm, measured in the dry state, are achieved using typical airbrush guns. After the coating, the electrode is dried and subsequently immersed for at least 2 hours at approximately 30° C. to 40° C. in a cross-linking agent solution of 2 to 4 ml glutaraldehyde in a water-acetone mixture. The cross-linking agent solution is then replaced for at least a further 2 hours. Subsequently, the implant is washed multiple times using distilled water and reductively fixed using a diluted solution of sodium cyanoborohydride, and washed multiple times using deionized water. After removal, the sample is dried for 24 hours at 50° C. in the drying cabinet. The electrical transmission properties of the electrode are not significantly impaired—up to a maximum layer thickness of 400 μm.

Integration of a Therapeutic Active Ingredient

As a supplement to the already existing intrinsic therapeutic effect of the hyaluronic acid, anti-inflammatory steroids, such as dexamethasone and/or dexamethasone sodium phosphate (DMNP) may be embedded in the coating, which are released into the surrounding tissue through the gradual degradation of the coating and/or through diffusion.

The polysaccharide layer is prepared in the same way as described in Example 2, but before the drying, the electrode is washed for an hour using 2 to 4 ml of a solution of 50 mg/ml DMNP. The drying is then performed without further washing steps, as described in Example 2.

Investigations of the Swelling Behavior

Different degrees of cross-linking, with otherwise identical reductive fixing, result in different swelling behavior of the hyaluronic acid. The swelling behavior may be determined gravimetrically, among other ways. Furthermore, the degree of cross-linking may also be determined through infrared spectroscopic analysis on cross-linked hyaluronic acid films. The reference for degradation may be produced through a GPC analysis, i.e., through molar mass determination of degraded hyaluronic acid, on eluents.

In order to determine the influence of cross-linking parameters on the cross-linking and therefore also on the swelling behavior, the parameters of temperature, water content, type of cross-linking agent, and cross-linking duration were varied. Hyaluronic acid films were cast and cross-linked to determine the correlation between swelling behavior and the cross-linking parameters.

### Examples 1 Through 8

### Experiments on the Swelling Behavior

The method according to Example 1 was divided into the following steps:
(a) preparing a 1% hyaluronic acid solution;
(b) pouring 3 ml 1% hyaluronic acid solution into Petri dishes having 4 cm diameter and subsequent drying;
(c) adding 4 ml cross-linking agent solution to the films at room temperature (20° C.), the cross-linking agent solution comprising 240 ml acetone, 80 ml 25% glutaraldehyde solution, and 1.6 ml 3 molar hydrochloric acid;
(d) cross-linking duration 20 hours, the cross-linking agent solution having been replaced after 4 hours;
(e) removal and washing with deionized water;
(f) adding 4 ml 2.2% NaBH₃CN solution;
(g) washing with deionized water;
(h) drying.

The further Examples 2 through 8 deviated as follows, with otherwise identical method control:

In Example 2, the cross-linking duration in step (d) was 4 hours without replacement of the cross-linking agent solution.

In Example 3, the cross-linking duration in step (d) was 2 hours without replacement of the cross-linking agent solution.

In Example 4, the cross-linking agent solution cited in step (c) additionally contained 20 ml deionized water.

In Example 5, the cross-linking agent solution cited in step (c) additionally contained 100 ml deionized water.

In Example 6, the cross-linking agent solution cited in step (c) contained 80 ml 25% formaldehyde solution instead of the glutaraldehyde solution.

In Example 7, the cross-linking in step (d) was performed at 30° C. and the cross-linking duration in step (d) was 6.5 hours, the cross-linking solution having been replaced after 1.5 hours.

In Example 8, the cross-linking in step (d) was performed at 30° C. and the cross-linking duration in step (d) was 7 hours, the cross-linking solution having been replaced after 2 hours.

After drying the cross-linked films, these were weighed and subsequently washed in deionized water for 30 minutes, blotted briefly and weighed again in order to determine the swelling behavior, which correlates with the degree of cross-linking.

The swelling factors determined may be inferred from the following table:

TABLE 1
swelling factors

Example

| | 1 | 2 | 3 | 4 | 5 | 6 | 7 | 8 |
|---|---|---|---|---|---|---|---|---|
| Swelling | 6 | 14 | 75 | 7 | 7 | 34 | 10 | 13 |
| factor | | | | | | | | |

The exemplary experiments on cross-linking led to the following conclusions:

The cross-linking duration has a significant influence on the degree of cross-linking, which is reflected in the swelling behavior. At a cross-linking duration of only 2 hours, hyaluronic acid films were obtained which were unstable and dissolved within a few hours in water. In contrast, at a cross-linking duration of 4 hours, stable hyaluronic acid films were obtained, which displayed a higher swelling factor than the films of the standard method, however. The water content of the cross-linking agent solution did not have a strong influence on the swelling factor, and therefore the degree of cross-linking, in the range examined. The use of formaldehyde instead of glutaraldehyde resulted in cross-linked hyaluronic acid films having a significantly higher swelling factor. This may possibly be attributed to the shorter chain length of the formaldehyde. The shorter cross-linking agent formaldehyde thus results in lightly cross-linked hyaluronic acid films. Cross-linking at a temperature of 30° C. and a cross-linking duration of 7 hours results in hyaluronic acid films having a somewhat higher swelling factor and therefore a lower degree of cross-linking.

We claim:

1. An implantable stimulation electrode for use with an implantable tissue stimulator, particularly a pacemaker, defibrillator, bone stimulator, or neurostimulator, the stimulation electrode comprising a metallic base body, optionally one or more intermediate layers applied to the base body, and a coating, which covers the base body and optionally the intermediate layers, to increase the tissue compatibility,
wherein the coating comprises a polysaccharide layer made of hyaluronic acid and/or hyaluronic acid derivatives.

2. The stimulation electrode according to claim 1,
wherein the hyaluronic acid and hyaluronic acid derivatives have an average molecular weight between 300,000 and 500,000 Dalton after a sterilization.

3. The stimulation electrode according to claim 2,
wherein the average molecular weight is between 380,000 and 420,000 Dalton.

4. The stimulation electrode according to claim 1,
wherein the polysaccharide layer has a composition such that the in vivo degradation of the polysaccharide layer is slowed from the outside in the direction of the base body of the stimulation electrode.

5. The stimulation electrode according to claim 4,
wherein an internal area of the polysaccharide layer is not degradable, at least not completely, within two years.

6. The stimulation electrode according to claim 5,
wherein the internal area is 3 to 50 μm thick.

7. The stimulation electrode according to claim 4,
wherein an external area of the polysaccharide layer is degradable in vivo within 100 days.

8. The stimulation electrode according to claim 7,
wherein the external area is 10 to 250 μm thick.

9. The stimulation electrode according to claim 4,
wherein the polysaccharide layer comprises at least two partial layers having different degradation behaviors, the degradation behavior within each partial layer being able to be fixed continuously changeably or constant over the partial layer.

10. The stimulation electrode according to claim 9,
wherein the polysaccharide layer comprises an internal partial layer which is degradable by not more than 20 weight-percent in vivo within 2 years.

11. The stimulation electrode according to claim 10,
wherein the internal partial layer is 3 to 50 μm thick.

12. The stimulation electrode according to claim 9,
wherein the polysaccharide layer comprises an external partial layer which is degradable by at least more than 50 weight-percent within 100 days in vivo.

13. The stimulation electrode according to claim 12,
wherein the external partial layer is 10 to 250 μm thick.

14. The stimulation electrode according to claim 4,
wherein a layer thickness of the coating is between 10-400 μm.

15. The stimulation electrode according to claim 14,
wherein the layer thickness is 50-120 μm.

16. The stimulation electrode according to claim 1,
wherein the coating contains dexamethasone and/or dexamethasone sodium phosphate (DMNP) in a concentration sufficient to produce a pharmacological effect.

17. The stimulation electrode according to claim 1,
wherein the hyaluronic acid or hyaluronic acid derivatives are components of the coating as individual substances, copolymers or block polymers made of hyaluronic acid and hyaluronic acid derivatives, or mixtures thereof.

18. The stimulation electrode according to claim 1,
wherein the polysaccharide layer is immobilized covalently or through physisorption on the surface of the stimulation electrode.

19. The stimulation electrode according to claim 1,
wherein the polysaccharide layer comprises an adhesion-promoting layer made of chitosan.

20. The stimulation electrode according to claim 19,
wherein the adhesion-promoting layer is 0.1 to 50 μm thick.

21. The stimulation electrode according to claim 1,
wherein the polysaccharide layer contains chitosan at least in partial areas or partial layers.

22. The stimulation electrode according to claim 21,
wherein a component of the chitosan in the total weight of the polysaccharide layer is not more than 50 weight-percent.
